(19) United States
(12) Patent Application Publication
(10) Pub. No.: US 2023/0392103 A1
(43) Pub. Date: Dec. 7, 2023

(54) TRANSPORT PACKAGING FOR A PLURALITY OF PETRI-DISHES AND BLISTER PACKAGING WITH A PLURALITY OF PETRI-DISHES

(71) Applicant: MERCK PATENT GMBH, DARMSTADT (DE)

(72) Inventors: Philippe RIVAT, Molsheim (FR); Mathieu ARRAULT, Molsheim (FR); Francois OBLINGER, Molsheim (FR); Stéphane OLIVIER, Molsheim (FR); Gael WAICHE, Molsheim (FR)

(73) Assignee: MERCK PATENT GMBH, DARMSTADT (DE)

(21) Appl. No.: 18/033,209

(22) PCT Filed: Oct. 20, 2021
(86) PCT No.: PCT/EP2021/079016

§ 371 Date: Apr. 21, 2023

(30) Foreign Application Priority Data
Oct. 23, 2020 (EP) 20306267.4

(51) Int. Cl. C12M 3/00; C12M 1/22; C12M 1/00; B01L 9/00

(52) U.S. Cl. CPC C12M 23/48; C12M 23/10; C12M 23/52; B01L 9/523

(57) ABSTRACT

The present application relates to a transport packaging (20) for a plurality of petri-dishes (P), comprising a shell-like main body (21) with a compartment (22) for holding the plurality of petri-dishes (P) in a parallel orientation, the compartment (22) being accessible from an opening (23) at one side of the main body (21), and a rack-like structure (24) formed in the compartment (22) for supporting the petri-dishes (P) in a fixed position during handling and configured to allow insertion/extraction of the petri-dishes (P) into/from the rack-like structure (24) in a direction.

### TECHNICAL FIELD

The present application concerns a transport packaging for a plurality of petri-dishes and a blister packaging with (including) a plurality of petri-dishes. The present application particularly concerns the field of testing in pharmaceutical and food processing and more particularly environmental monitoring of clean or ultra clean processing areas. It is also applicable to other processing situations where cleanness of a processing area or environment is to be determined and monitored, for example in the field of semiconductor, electronics or aircraft manufacturing.

### BACKGROUND

In order to monitor environmental conditions in closed processing areas of the above type it is common practice in passive air sampling to place one or more media plate/plates in an activity zone of the production area and expose them to the surrounding air such that they can capture the maximum amount of particles in the surrounding air. Larger particles tend to settle faster on the plates due to gravitational force. Smaller particles take some time in settling due to factors such as air currents. Media plates work best in still areas. The microorganisms from the air may settle on the media plates alone or in colonies.

In active monitoring of air in production areas a microbial air sampler is used to force air into or onto a collection medium over a specified period of time. The collection medium can be a common petri-dish, for example including a nutrient agar-based test media or other suitable test media depending on the need.

The collection media, for example in the form of the media plates or settle plates (the terms will be used interchangeably in this specification), have to be transferred repeatedly into the production area and removed therefrom for further handling and evaluation. This is commonly done in a manual process where one or more plates or petri-dishes are conveyed manually through a sterile transfer port into and from the interior of the production area. However, the manual handling of the petri-dishes involves a high risk of contamination when handling the media plate after a lid, cover or seal has been opened, and a non-secure transfer of the media plates during the introduction, installation and removal, in particular when a plurality of them are handled at once.

The common petri-dishes are not particularly suitable for automated handling individually or in batches as they typically consist of a media plate holding the nutrient media and a lid or cover releasably covering the plate. It is difficult for automated handling equipment to securely grip, hold and transfer the smooth cylindrical surfaces of the media plate and/or lid/cover, and there is a high risk that lids are inadvertently opened, displaced or removed from their media plates during handling, thereby compromising the detection result.

US 2002/053525 A discloses a cassette arrangement for accommodating petri-dishes or the like. The arrangement includes a box-shaped cassette housing designed to be stackable and having a slotted opening on one lateral side for inserting or removing the petri-dish from an interior space of the housing. The box-shaped cassette housing is stackable and has finger-operated means arranged on an opposite end side for pushing the petri-dish in the cassette housing out at least partially through the opening. While this cassette arrangement provides for a safe transport of the petri-dish held in the interior space of the cassette housing, it is not useful for automated handling as it is specifically directed to a manual operation by a finger in order to at least release the petri-dish from the housing. Further, the stacking capability is not specifically reliable and likewise not suitable for automated handling of a stack of cassette housings.

What is desired is an at least partly or preferably fully automated process that does not involve human handling steps for introducing, installing and removing the media plates into and from the production areas.

It is furthermore preferable to provide means for such a partly or fully automated process with which it is possible to use standard media plates available on the market, preferably the so-called petri-dishes.

In addition, any solution should preferably facilitate the traceability of samples during the processes.

The application accordingly aims at providing a transport packaging for a plurality of petri-dishes and a blister packaging with a plurality of petri-dishes, which allows secure handling of petri-dishes or settle plates used for microbiological air sampling in a classified or non-classified environment reducing the risk of false positive and final product contamination, which allows a full automation of the air testing from storage, transportation to sampling area, sampling, counting, waste management for passive or active microbiological air sampling in a fully automated, a partially automated or a manual process, i.e. the manual handling should not be excluded.

### SUMMARY

According to the present application this object is solved by providing a transport packaging for a plurality of petri-dishes as defined in claim 1 and a blister packaging with a plurality of petri-dishes as defined in claim 12.

The present application specifically provides a transport packaging for a plurality of petri-dishes, comprising:
a shell-like main body with a compartment for holding the plurality of petri-dishes in a parallel orientation, the compartment being accessible from an opening at one side of the main body; and
a rack-like structure formed in the compartment for supporting the petri-dishes in a fixed position during handling and configured to allow insertion/extraction of the petri-dishes into/from the rack-like structure in a direction. Preferably, said direction is essentially perpendicular to the axis of the rack-like structure.

Preferably, the rack-like structure is integrally formed with the compartment.

Preferably, the main body comprises an external stand for placing the transport packaging in a defined posture, preferably in an essentially vertical and/or an essentially horizontal orientation.

Preferably, the rack-like structure is formed with spacers for individually supporting the petri-dishes with a clearance from each other and/or from lateral walls of the main body to allow gripping of the Petri-dishes at their top and bottom sides and/or their peripheral wall from the side of the access opening and removal/insertion through the opening.

Preferably, the rack-like structure is formed to hold a stack of petri-dishes without separation from each other so as to block direct removal and allow movement along the direction of the stack, and includes a defined dispensing portion at the end of the stack that allows insertion/extraction of the petri-dishes from the opening in the direction. Preferably, said direction is essentially perpendicular to the axis of the rack-like structure.

Preferably, the rack-like structure is formed to support the petri-dishes in one or more row/rows.

Preferably, the rack-like structure is formed to support the petri-dishes in at least two rows in a parallel and spaced apart manner or in a staggered manner.

Preferably, the main body includes a dedicated storage compartment to receive a folded bag for accommodating, in an unfolded state, the shell-like main body.

Preferably, the main body includes a manually deformable zone for selectively reducing an internal volume of the compartment without interfering with the Petri-dishes accommodated in the compartment.

Preferably, the shell-like main body is formed as a one-piece blister cavity, preferably formed from a formable sheet or web material, and further preferably by thermoforming or cold-forming or a combination thereof.

Preferably, the opening of the shell-like main body for access to the compartment is configured to be sealed by a gas barrier film, preferably at least partially transparent, to form a blister-like packaging.

The present application also provides a blister packaging with a plurality of petri-dishes, comprising:
a transport packaging according to the present application, wherein the plurality of petri-dishes are accommodated in the rack-like structure in the compartment in a sterile environment; and
a gas barrier film, preferably at least partially transparent, sealing the opening of the shell-like main body.

Preferably, the blister packaging further includes a folded bag for accommodating, in an unfolded state, the shell-like main body.

Preferably, the shell-like main body and/or the gas barrier film are configured such that one or more of a label, a bar-code, a RFID chip, a humidity indicator, a sterility indicator inside the packaging can be detected by one or more of naked eyes, a camera, a reader and a sensor without opening the packaging.

### BRIEF DESCRIPTION OF THE DRAWINGS

Preferred embodiments will now be described with respect to the attached exemplary schematic drawings in which:

FIG. 1 is a schematic top-view of a transport packaging according to an embodiment with a plurality of petri-dishes.

FIG. 2 is a schematic side-view of the transport packaging of FIG. 1.

FIG. 3 is a perspective view of a transport packaging according to an embodiment with a plurality of petri-dishes.

FIG. 4 is a bag for accommodating the transport packaging.

FIG. 5 is a perspective and partially cut-away view of the transport packaging of FIG. 3.

FIG. 6 is a perspective view of a transport packaging according to a further embodiment.

FIG. 7 is a schematic top-view of a transport packaging with petri-dishes in a staggered arrangement according to a further embodiment.

FIG. 8 is a perspective and partially cut-away view of the transport packaging of FIG. 7 interacting with a gripper.

FIG. 9 is a perspective view of a transport packaging according to a still further embodiment.

FIG. 10 is a perspective and partially cut-away view of the transport packaging of FIG. 9 cooperating with a gripper.

FIG. 11 is a schematic cross-sectional view of a transport packaging according to a still further embodiment.

FIG. 12 is a schematic cross-sectional view of the transport packaging of FIG. 11.

FIG. 13 is a representation showing a sequence of steps for producing a blister packaging with petri-dishes.

FIG. 14 is an example of a process of handling petri-dishes during environmental monitoring of a clean processing area.

### DETAILED DESCRIPTION

For the purposes of the present application, terms such as “horizontal”, “vertical”, “perpendicular”, and similar terms are—if not already explicitly indicated—considered to be “essentially horizontal”, “essentially vertical”, “essentially perpendicular”, provided that this does not negatively affect functionality. Preferably, the term “essentially” is to denote a deviation of at most 10°, more preferably of at most 5°, even more preferably of at most 4° or 3°, still even more preferably of at most 2° or 1° from being horizontal, vertical, and perpendicular, respectively.

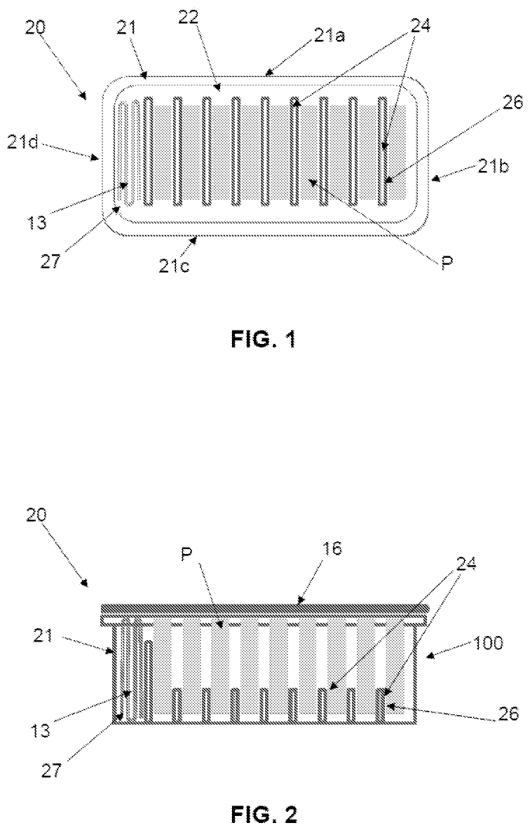

An embodiment of a transport packaging for a plurality of petri-dishes or settling plates according to the present application is schematically shown in FIGS. 1 and 2.

The transport packaging 20 according to the present application is in particular designed so as to be compatible with standard petri-dishes or standard nutrition or settling plates used for environmental monitoring that can be introduced into a environment of the appropriate cleanliness grading (for example pharmaceutical class A classified space that satisfies European Medicines Agency (EMA) and PIC/S requirements to meet ISO 5 measured via airborne 0.5 Linn particulate, ISO 4.8*measured via airborne ≥5.0 μm particulate in the in-operation and at-rest states, airborne viable microorganisms <1 colony forming unit (CFU) per cubic meter; these spaces are normally unidirectional flow with a suggested air velocity of 0.36-0.54 meters per second) and subsequently re-used for the incubation and evaluation procedures.

The transport packaging 20 has a shell- or tray-like main body 21 in an overall parallel-epiped or box-like outer shape and with a peripheral rim surrounding an opening 23 and an internal compartment 22 for holding the plurality of petri-dishes P in a parallel orientation. The compartment 22 is accessible through the opening 23 on the top side of the main body 21.

A rack-like structure 24 is formed in the compartment 22 for supporting the petri-dishes P in individual separated sections in a fixed and defined position during handling and which is configured to allow insertion/extraction of the petri-dishes P into/from the sections of the rack-like structure 24 in a specific direction, normally perpendicular to the plane defined by the top-side of the rim surrounding the opening 23. It is noted that the rim surrounding the opening 23 may also be just formed by the walls of the tray-line body 21.

As shown in various of the figures, the rack-like structure 24 is preferably integrally formed or molded with the compartment 22 as a one-piece structure of the shell-like main body 21. It may, however, be formed as a separate element in the form of an insert that is inserted and held within the compartment 22.

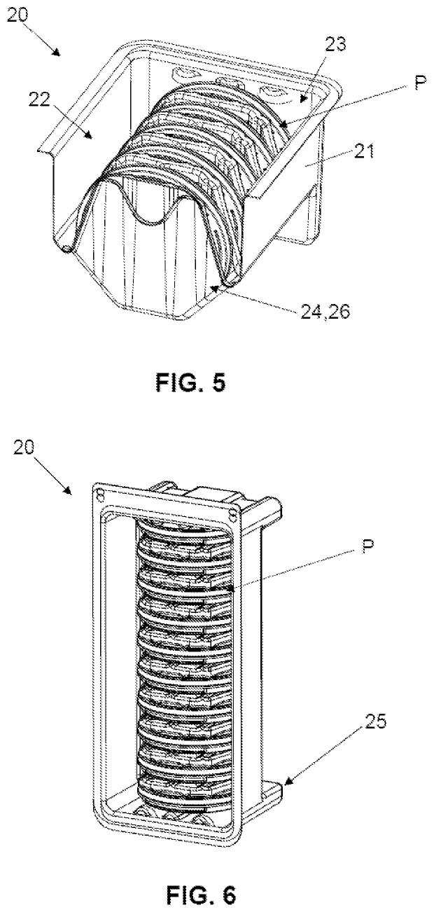

As shown in FIGS. 5 and 6 in particular, the main body 21 may have an external stand for placing the transport packaging 20 in a defined posture, preferably in an essentially vertical orientation and/or in an essentially horizontal orientation as shown in these figures. The stand 25 is preferably integrally formed with the shell-like main body on its outer periphery and can be in a form of a protrusion on one side surface of the main body increasing the distance of supporting points to increase the stability. Several external stands may be provided to allow placing of the transport packaging in different orientations so as to present the petri-dishes on the desired posture or orientation in the different steps of the process (for example loading in an insulator, conveying to a collection area, setting up for the distribution and dispensing of media for sampling, collection and removal from the insulator, incubation etc.).

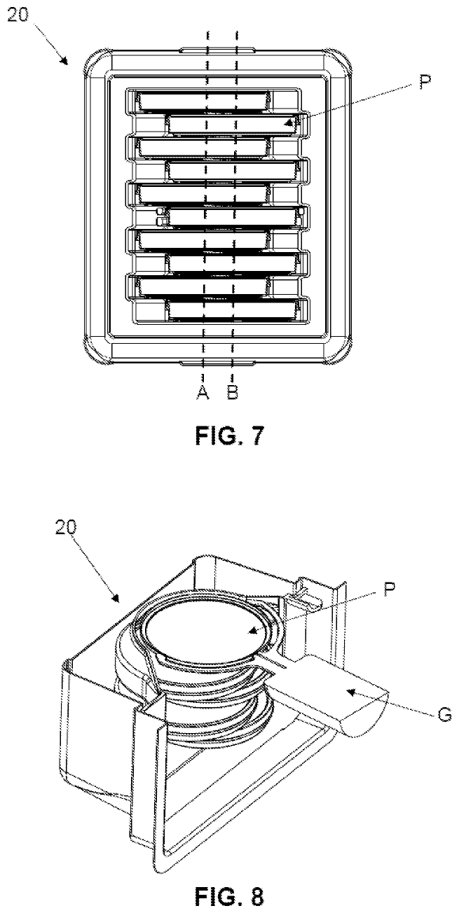

The rack-like structure 24 is formed with the sections in the form of spaces 26 for individually supporting the petri-dishes P with a clearance from each other and/or from lateral walls 21a to d of the main body 21 to allow gripping of the petri-dishes individually at their top and bottom sides and/or at their peripheral wall from the side of the access opening 23 and for allowing the unimpeded direct removal/insertion through the opening 23. The clearances are designed so that the gripping may be effected manually and/or by an automated mechanical gripper. The rack-like structure 24 may be formed so as to support the petri-dishes P in one or more row/rows A, B (see FIG. 7 as an example). Preferably, in such arrangement the petri-dishes P of each row are then essentially aligned along the axis of the respective row. In one variant the rack-like structure 24 is formed to support the petri-dishes P in at least two rows A, B in a parallel staggered manner (see FIG. 7). The staggered arrangement increases the access clearance on at least one lateral side of each petri-dish without necessarily increasing the overall size of the packaging. In another variant (not shown) two or more stacks of mutually parallel petri-dishes can be arranged such that the stacks are parallel and spaced apart from each other.

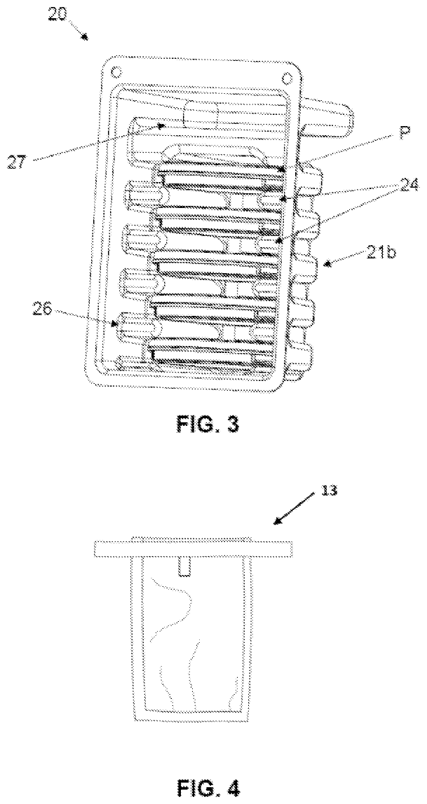

In a preferred embodiment the main body 21 includes a dedicated storage compartment 27 sized to receive a folded bag 13 for accommodating, in an unfolded state of the bag 13, the shell-like main body 21 (see FIGS. 3 and 4). In this embodiment the dedicated storage compartment 27 is located at one axial end of the shell-like main body in the compartment 22, and the bag may include the zip-lock or other easily openable and re-sealable closing features.

The provision of the storage compartment 22 and the possibility to receive the unfolded bag 13 facilitates the handling during various steps of the process after initial opening of a gas barrier film closing the packaging as the essential elements are provided together in the appropriate size and quality. The bag in the dedicated storage compartment 27 is empty and preferably sterilized. It may be used, for example, to protect the petri-dishes in the transport packaging from contamination after the environmental testing when transported out from the classified area that is to be examined and during transfer to a microbiological lab site. The dedicated space or another dedicated space may be provided for accommodating other elements required in the process like irradiation or humidity indicators, desiccant, testing reagents.

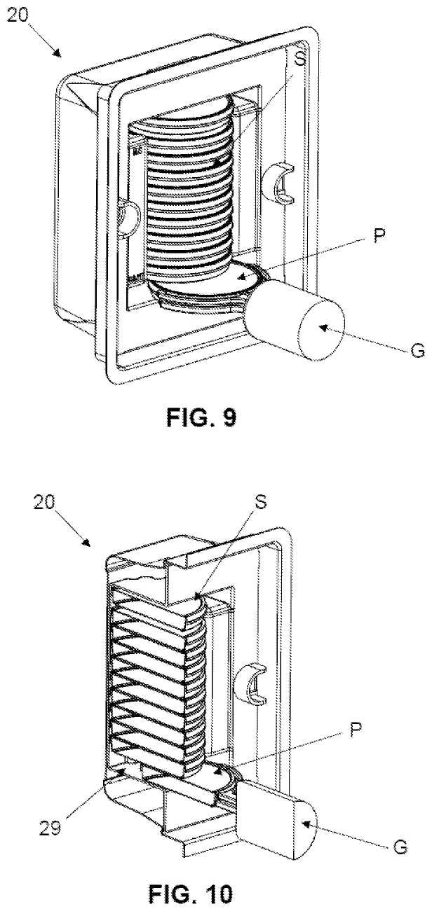

In a preferred embodiment shown in FIG. 9 the rack-like structure 24 may be formed to hold a stack S of petri-dishes P without separation from each other in an accommodating space that blocks direct removal towards the side of the opening 23 in the direction perpendicular to the plane defined by the peripheral rim surrounding an opening 23 and allows a guided movement only along the direction of the stack S under the influence of gravity at least over a defined extension. The accommodating space may include the defined dispensing portion 29 at one or both axial ends of the stack S that allow(s) insertion/extraction of the petri-dishes P one after the other from the opening 23 in the direction. Accordingly, the plates arranged in the stack or pile are blocked in the compartment 22 except the plate at the end of the pile or stack (the lower end in FIG. 10). The rack-like structure 24 thus forms a kind of a magazine with the possibility of extraction of the lowermost plate by means of an automated gripper G. The plates may be re-supplied into the compartment at the upper end.

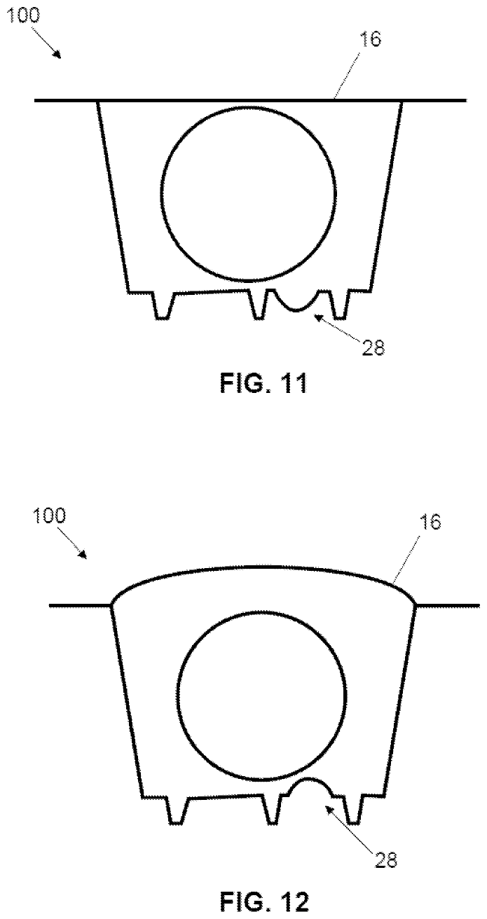

In a preferred embodiment shown in FIGS. 11 and 12 the main body 21 may include a manually deformable zone 28, for example at the bottom of the shell-like main body 21, which allows selective reducing of an internal volume V of the compartment 22 without interfering with the petri-dishes P accommodated within the compartment 22. The deformable zone 28 allows voluntary sinking by pressing to reduce the volume of the internal space of the compartment of the main body, thus creating an over-pressure as long as the shell-like main body is tightly closed at the opening 23, thus bulging out a barrier-gas film sealing the opening. With this aspect it will be possible, before use and exposure to gases, to easily test the integrity of the packaging without the risk of affecting the properties of the media in the petri-dishes, including the fertility etc. It is pointed out that the feature of the manually deformable zone 28 at a suitable portion of the shell-like main body 21 can be applied to all variants and embodiments described in this application even though it is described here in connection with FIGS. 11 and 12 in a schematic manner only.

The shell-like main body 21 may be formed as a one-piece self-supporting blister cavity or part, preferably formed from a foldable sheet or web material, and may be further preferably formed by thermoforming or cold forming or by a combination thereof.

The shell-like main body 21 may be formed from plastic material and may be designed for multi-use (including intermediate sterilization) or one-time use (i.e. as disposable product).

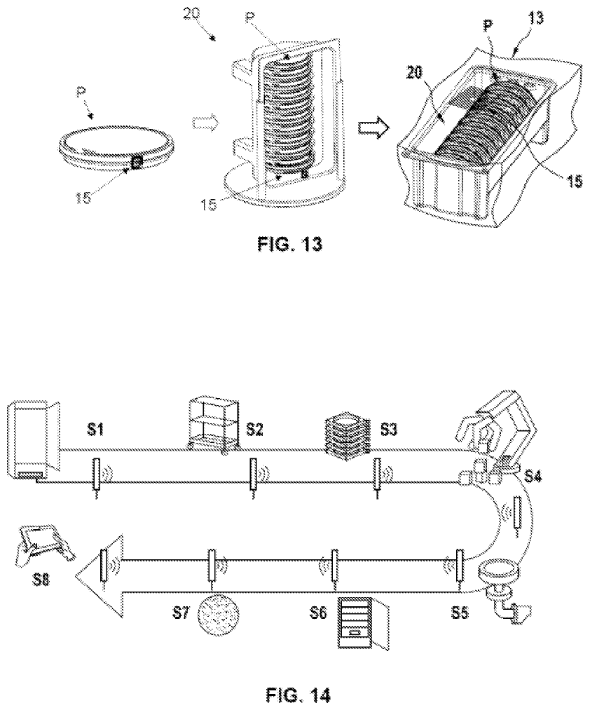

As a result of a manufacturing or collating process as schematically indicated in FIG. 13 the present application may provide a blister packaging 100 with the plurality of petri-dishes P comprising a transport packaging 20 according to the present application, bearing the plurality of petri-dishes P accommodated in the rack-like structure 24 in the compartment 22 in a sterile environment, and a gas barrier film 16 sealing the opening 23 of the shell-like main body 21.

The opening 23 of the shell-like main body 21 for access to the compartment 22 is configured to be sealed by the gas barrier film 16, that is preferably at least partially transparent, to form a blister-like packaging 100 which remains sterile until opened by removing the gas barrier film 16. The shell-like main body 21 may be at least partially transparent, too. The transparency of the gas barrier film and/or of the shell-like main body allows scanning, reading or detecting of traceability information of each petri-dish or plate without opening the packaging and without taking it out of its receptacle in the rack-like structure. The traceability information may be provided in the form of one or more of a label, a bar-code, a RFID chip, a humidity indicator, a sterility indicator that can be detected by one or more of naked eyes, a camera, a reader and a sensor. This avoids the risk of damage to the plate and accidental contamination.

Traceability elements on labels in the form of bar codes, data matrix or RFID may be accommodated and provided on the bag for the packaging or on the sections for accommodating the individual petri-dishes and/or directly on the petri-dishes.

Based on the traceability elements all critical process data may be recorded and linked through an integrated RFID-type system or via an external system, for example cloud-based. Such a process is schematically shown in FIG. 14 and includes a number of typical stations or stages of the process where specific data is collected and recorded like the storage stage S1 (time/date of entrance, storage temperature, moisture level, expiry date management, fifo management), transportation stage S2 (duration and temperature), grouping stage S3 (position or group to which the individual petri-dishes belong), transfer stage S4 (location and time), air sampling stage S5 (duration, location, time/date, link of the ID of the plate with the ID of the rack ID), incubation stage S6 (time/date of entrance, real temperature, moisture level, O2 level), counting stage S7 (time/date and counting results) where counting takes place in order to determine the number of contaminants. At stage S8 all the data are exported and transferred to a storage from where they can be accessed and further processed.

The traceability can be realized externally with dedicated RFID reader systems connected to a central system, but the RFID (or data matrix) reader can also be driven directly from the air monitoring system which allows full traceability of the process plate including:
positive detection of the processed plate using data matrix reader;
positive detection of the plate storing rack (e.g. blister) using data matrix or RFID;
allowing read and write information in the RFID tag of the blister containing the plates;
each blister tag may contain the following information: batch number, location site number, set number, ID of the plate, position of the plate inside the blister;
time when the plate was placed and removed from the isolator;
hour of the sampling, conformity of the air monitoring parameters, operator, events during the presence of the blister in the sampling environment.

1. A transport packaging (20) for a plurality of petri-dishes (P), comprising:
a shell-like main body (21) with a compartment (22) for holding the plurality of petri-dishes (P) in a parallel orientation, the compartment (22) being accessible from an opening (23) at one side of the main body (21); and
a rack-like structure (24) formed in the compartment (22) for supporting the petri-dishes (P) in a fixed position during handling and configured to allow insertion/extraction of the petri-dishes (P) into/from the rack-like structure (24) in a direction.

2. The transport packaging (20) for a plurality of petri-dishes (P) according to claim 1, wherein the rack-like structure (24) is integrally formed with the compartment (22).

3. The transport packaging (20) for a plurality of petri-dishes (P) according to claim 1, wherein the main body (21) comprises an external stand (25) for placing the transport packaging (20) in a defined posture, preferably in an essentially vertical and/or an essentially horizontal orientation.

4. The transport packaging (20) for a plurality of petri-dishes (P) according to claim 1, wherein the rack-like structure (24) is formed with spacers (26) for individually supporting the petri-dishes (P) with a clearance from each other and/or from lateral walls (21a-d) of the main body (21) to allow gripping of the Petri-dishes (P) at their top and bottom sides and/or their peripheral wall from the side of the access opening (23) and removal/insertion through the opening (23).

5. The transport packaging (20) for a plurality of petri-dishes (P) according to claim 1, wherein the rack-like structure (24) is formed to hold a stack (S) of petri-dishes (P) without separation from each other so as to block direct removal and allow movement along the direction of the stack (S), and includes a defined dispensing portion (29) at the end of the stack (S) that allows insertion/extraction of the petri-dishes (P) from the opening (23) in the direction.

6. The transport packaging (20) for a plurality of petri-dishes (P) according to claim 1, wherein the rack-like structure (24) is formed to support the petri-dishes (P) in one or more row/rows (A,B).

7. The transport packaging (20) for a plurality of petri-dishes (P) according to claim 6, wherein the rack-like structure (24) is formed to support the petri-dishes (P) in at least two rows (A,B) in a parallel and spaced apart manner or in staggered manner.

8. The transport packaging (20) for a plurality of petri-dishes (P) according to claim 1, wherein the main body (21) includes a dedicated storage compartment (27) sized to receive a folded bag (13) for accommodating, in an unfolded state, the shell-like main body (21).

9. The transport packaging (20) for a plurality of petri-dishes (P) according to claim 1, wherein the main body (21) includes a manually deformable zone (28) for selectively reducing an internal volume (V) of the compartment (22) without interfering with the Petri-dishes (P) accommodated in the compartment (22).

10. The transport packaging (20) for a plurality of petri-dishes (P) according to claim 1, wherein the shell-like main body (21) is formed as a one-piece blister cavity, preferably formed from a formable sheet or web material, and further preferably by thermoforming or cold-forming or a combination thereof.

11. The transport packaging (20) for a plurality of petri-dishes (P) according to claim 1, wherein the opening (23) of the shell-like main body (21) for access to the compartment (22) is configured to be sealed by a gas barrier film (16), preferably at least partially transparent, to form a blister-like packaging (100).

12. A blister packaging (100) with a plurality of petri-dishes (P), comprising:
a transport packaging (20) according to claim 1, wherein the plurality of petri-dishes (P) are accommodated in the rack-like structure (24) in the compartment (22) in a sterile environment; and
a gas barrier film (16), preferably at least partially transparent, sealing the opening (23) of the shell-like main body (21).

13. The blister packaging (100) according to claim 12, further including a folded bag (13) for accommodating, in an unfolded state, the shell-like main body (21).

14. The blister packaging (100) according to claim 12, wherein the shell-like main body (21) and/or the gas barrier film (16) are configured such that one or more of a label, a bar-code, a RFID chip, a humidity indicator, a sterility indicator inside the packaging (100) can be detected by one or more of naked eyes, a camera, a reader and a sensor without opening the packaging (100).
